(19) United States
(12) Patent Application Publication
(10) Pub. No.: US 2010/0190235 A1
(43) Pub. Date: Jul. 29, 2010

(54) METHODS FOR DESIGNING AND OPERATING PHOTOBIOREACTOR SYSTEMS

(76) Inventors: Christopher S. Schuring, Penryn, CA (US); J. Kyle McCue, San Jose, CA (US)

Correspondence Address: WILSON, SONSINI, GOODRICH & ROSATI, 650 PAGE MILL ROAD, PALO ALTO, CA, 94304-1050, US

(21) Appl. No.: 12/582,698

(22) Filed: Oct. 20, 2009

Related U.S. Application Data
(60) Provisional application No. 61/106,966, filed on Oct. 20, 2008.

(51) Int. Cl. C12N 1/00

(52) U.S. Cl. 435/243

(57) ABSTRACT

The invention may include methods of configuring and operating a photobioreactor system with modular photobioreactor units or blades. The invention may also include apparatus and methods of controlling the photobioreactor system and optimizing growth conditions through a support structure and backplane interface.

### CROSS-REFERENCE

This application claims the benefit of U.S. Provisional Application No. 61/106,966, filed Oct. 20, 2008, which application is incorporated herein by reference.

### BACKGROUND OF THE INVENTION

Carbon dioxide concentrations in the atmosphere are increasing. The burning of fuel produces carbon dioxide, which is released to the atmosphere and adds about 6 gigatons of carbon to the atmosphere each year. Carbon dioxide concentrations in the atmosphere have risen from about 270 parts per million (0.026%) before the industrial age to about 300 parts per million (0.036%) by 2006, a 41% increase over pre-industrial values. The increased concentration of this greenhouse gas in the atmosphere influences the earth's radiation balance. Carbon sequestration is a process of utilizing modern technologies to remove carbon dioxide from the source. Many companies involved in carbon dioxide sequestration are looking for ways to store carbon dioxide in geologic formations.

At the same time, the commercial potential of producing biomass products by photosynthesis techniques using simple plant matter, such as algae, blue green bacteria, and seaweed, has been recognized. Such techniques seek to harness the ability of photoautotrophic organisms to utilize sunlight and carbon dioxide to produce biomass products. Methods involving open-systems for cultivation of photoautotrophic organisms have been attempted. However, such methods have been impractical for numerous reasons, including contamination, low yield, loss of water, and inefficient use of light. Closed-system photobioreactors have been designed to address these limitations. However, these systems are not readily increased in scale and are not space-efficient.

A need exists for photobioreaction methods and apparatus that receives carbon dioxide and provides scalable and efficient methods for the growth of photoautotrophic organisms.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 shows a method of receiving harmful emissions and using them to provide beneficial applications.

FIG. 2 shows an example of some inputs and outputs to a photobioreactor system.

FIG. 3 shows a blade channel rail for pipe support in accordance with one embodiment of the invention.

FIG. 4A shows an example of support structure components.

FIG. 4B shows an example of a support structure.

FIG. 5 shows an example of how a photobioreactor unit can be mounted onto a rack from a top view.

FIG. 6 shows an alternate example of how a photobioreactor unit can be mounted onto a rack from a top view.

FIG. 7 shown an example of a control system in accordance with one aspect of the invention.

FIG. 8 shows an example of an optimization method in accordance with one aspect of the invention.

### DETAILED DESCRIPTION OF THE INVENTION

While preferable embodiments of the invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention.

FIG. 1 shows a method of receiving harmful emissions and using them to provide beneficial applications. For instance, a photobioreactor can receive one or more harmful greenhouse gases, such as carbon dioxide (CO₂) and use it to grow a photoautotrophic organism, such as algae, blue green bacteria, seaweed, or any other type of vegetation or organism growing through photosynthesis. A photobioreactor may be beneficially recycling emissions as opposed to merely sequestering them. The photoautotrophic organism or biomass products associated with the organism may have beneficial uses. Such an arrangement may enable an interaction between a CO₂ producing party, such as a power plant, waste energy plant, ethanol plant, petroleum refinery, manufacturing facilities and other emitters of greenhouse gases, and a market for biomass products. For instance, a CO₂ producing party may interact with an algae-growing party, which may interact with another party that may receive algae or algae-based products.

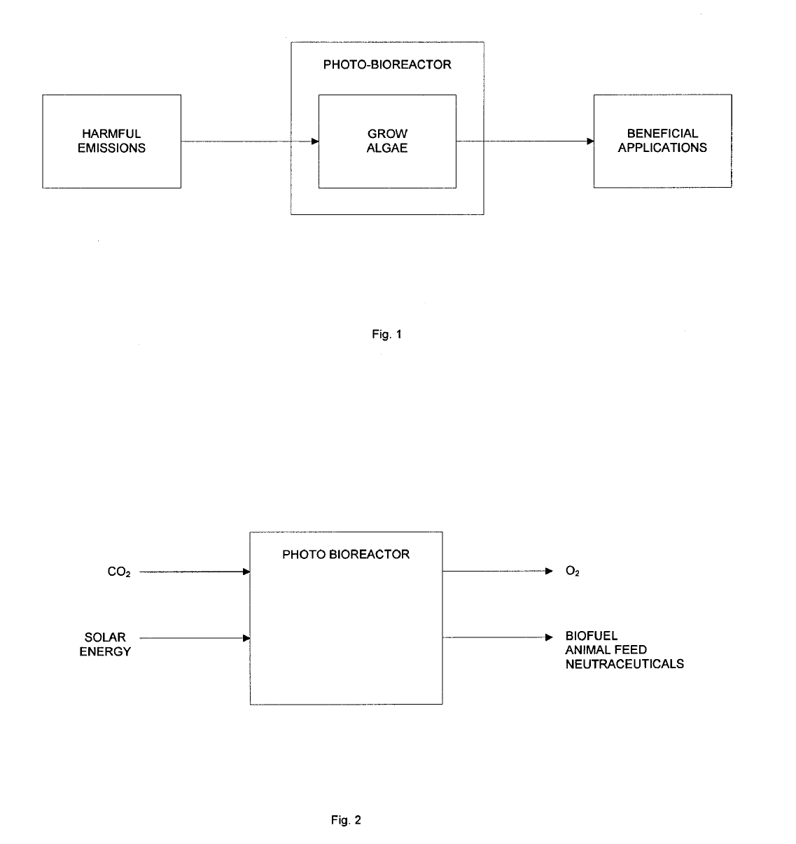

FIG. 2 shows an example of some inputs and outputs to a photobioreactor system. For instance, a photobioreactor may receive CO₂ to grow algae or other photoautotrophic organism such as algae, and solar energy to provide energy to run the system. For instance, solar power may generate electricity needed for operating a control system. Some of the products from the photobioreactor system include O₂ from the algae, and various algae-based products, which may include biofuel, animal feed, pharmaceuticals, and neutraceuticals (e.g., astaxanthin). Any form of photoautotrophic organism may be grown in a photobioreactor in addition to algae, and any discussion of algae or other cultures grown herein may be applied to any form of growth involving photosynthesis. Various types of algae may be grown, including, but not limited to, spirulina or chlorella.

A photobioreactor may have a bladelike configuration that may be supported by a support structure, such as a rack. A blade can be mounted with a vertical orientation. In some embodiments, the blade configuration can be supported by a rack. In some embodiments, the blade configuration can be supported from above or below or a combination thereof. One or more blades can be mounted substantially parallel to one another. In alternate configurations, the blades can be mounted to be perpendicular to one another or to have any orientation with respect to one another. In some embodiments, a photobioreactor unit may refer to a blade, and a rack with a plurality of blades may be referred to as a group of photobioreactor units. In other embodiments, a photobioreactor unit may refer to a grouping, such as a rack of blades.

A blade configuration may be one example of a photobioreactor device. Photobioreactor devices can include various shapes or configurations that enable the growth of a photoautotrophic organism, such as algae. Any discussion of blades may also apply to photobioreactor devices of various configurations.

In one example blade may have a substantially vertical orientation with a plurality of pipes with a substantially horizontal configuration. Such pipes may be connected to one another. In other examples, blades may be mounted with a substantially horizontal or angular orientation with a plurality of pipes that may be oriented in any configuration or direction. A benefit of such a blade configuration may be that it has a small footprint. A method may be provided of increasing or optimizing algae growth per square foot of land. A blade design may be created to increase or optimize algae growth for a given area and type of algae. The blade design may consider environmental conditions and/or energy input to create a desirable growing condition.

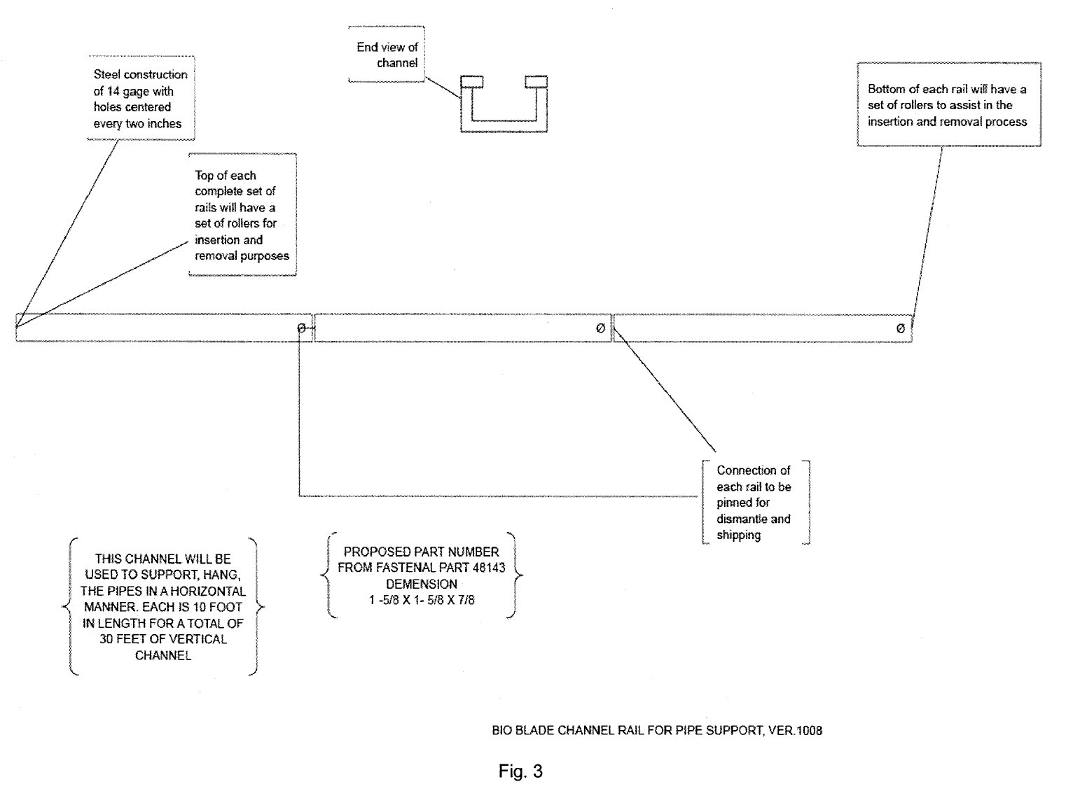

FIG. 3 shows a blade channel rail for pipe support in accordance with one embodiment of the invention. A blade channel rail can provide support to a blade. A channel rail can contain one or more connection with a pin configuration. A channel rail can be assembled or disassembled for transport, and can include configurations that provide simple assembly and disassembly while providing the necessary structural support.

In accordance with some embodiments of the invention, a method of assembling a photobioreactor may include receiving various photobioreactor and support components. Support components may be assembled by fastening components together with various fastening mechanisms such as clamps, adhesives, screws, bolts, or by using a lock and groove type mechanism or any other means of connecting components as is known in the art. In some embodiments, the components may be readily disassembled.

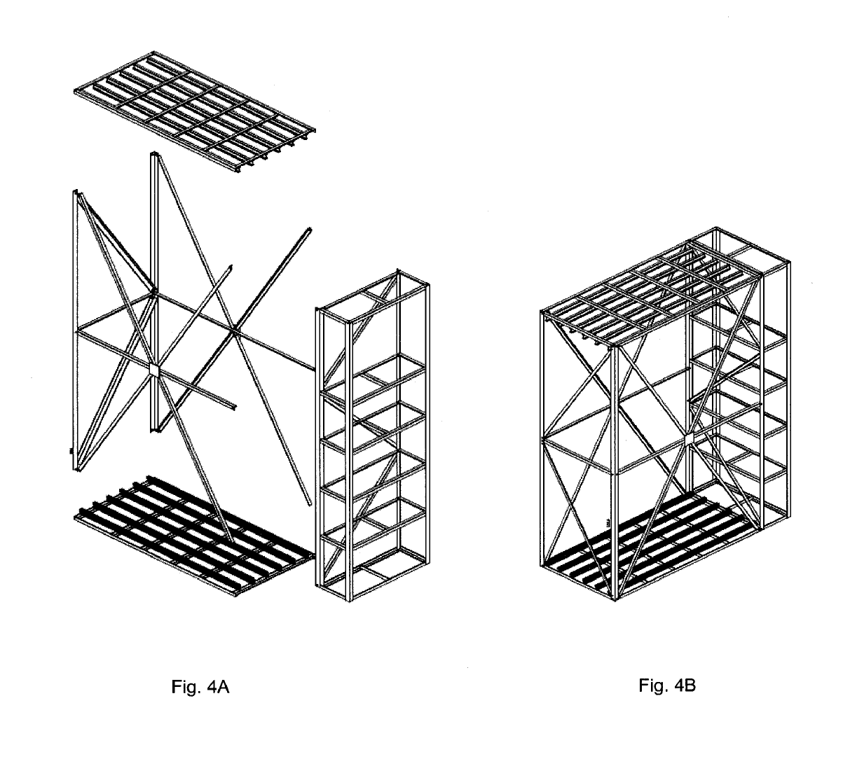

FIG. 4A shows an example of support structure components. For example, various components of a support structure may be pre-assembled, and then the components may be assembled on site. FIG. 4B shows an example of a support structure. The support structure may be assembled from the various components.

In some implementations, a method of configuring a photobioreactor may include receiving photobioreactor and support components at a desired location, assembling the components at the location, then making any adjustments to component configuration as desired for the operation of the photobioreactor. In some cases, the components may be disassembled and taken to another location.

In some cases, different components may be swapped in or out to provide desired utility. For example, different blades may grow different kinds of algae. In some embodiments, different blade configurations may be provided that optimize the growth of different types of algae. For example, for certain vegetative growths, pipes with a greater diameter may be preferred.

When environmental conditions change, it may be desirable to swap in blades with algae that prefer the new environmental conditions. For example, depending on the time of year, a photobioreactor may receive more sunlight, and certain algae may grow better with greater sunlight strength or longer periods of exposure to sunlight. In such situations, it may be beneficial to swap in blades with such types of algae.

Sunlight is one example of an environmental condition, and may change seasonally, temporally (time of day), or meteorologically (based on weather). Another example of an environmental condition is temperature, which may also vary depending on season, time of day, or weather. Natural environmental conditions may or may not have more effect, depending on whether the photobioreactors are set up outdoors or indoors (or in a similarly contained environment). If photobioreactors are operating indoors, the environmental conditions indoors may be controlled.

Various components of a photobioreactor or supporting structure may be swapped in or out as well. For example, if a photobioreactor is exposed to a colder temperature, additional heaters may be provided. In another example, depending on time of day or type of algae, different types of supplementary light sources may be swapped in or out. Thus, modularity may be provided by the organism growth reservoirs, such as the blades, as well as the supporting structures and components thereof.

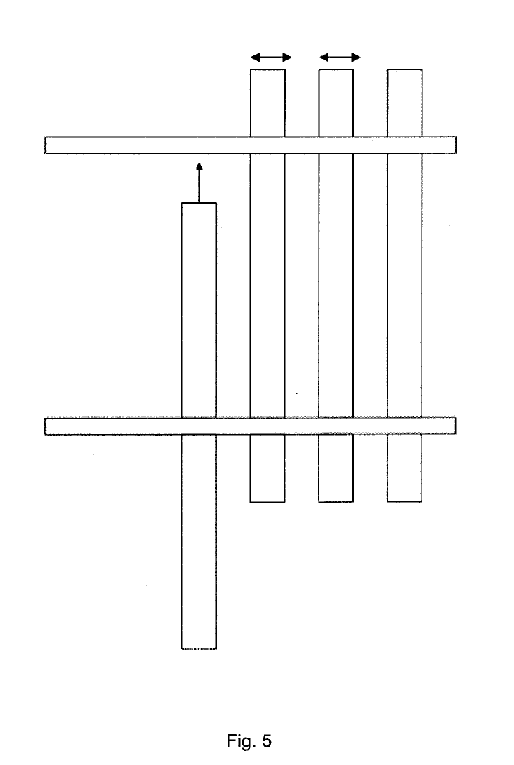

FIG. 5 shows an example of how blades can be mounted onto a rack from a top view. In some embodiments of the invention, a rackable blade may be mounted onto a rack by sliding in along a longitudinal axis with respect to the blade. For instance, a rack may have predetermined slots or rails that may provide a location through which a blade may slide. The predetermined slots or rails may be fixed such that once a blade slides into the rack, it may not move along any other direction. In some embodiments, once a blade is placed on the rack, it may be locked into place. A rackable blade may slide into a rack by using any sort of mechanism known in the art including, but not limited to, rails, configurations utilizing ball bearings, wheels, or guides.

In some implementations, a backplane may be provided. The backplane may include supporting components, pumps, tanks, or control and monitoring instrumentation, to enable the growth of cultures within the blade. When a blade slides into a rack, it may interface with the backplane. For example, a rack may be configured so a blade slides in a perpendicular direction to the backplane. In some embodiments, a blade may slide in at an angle to the backplane and rack.

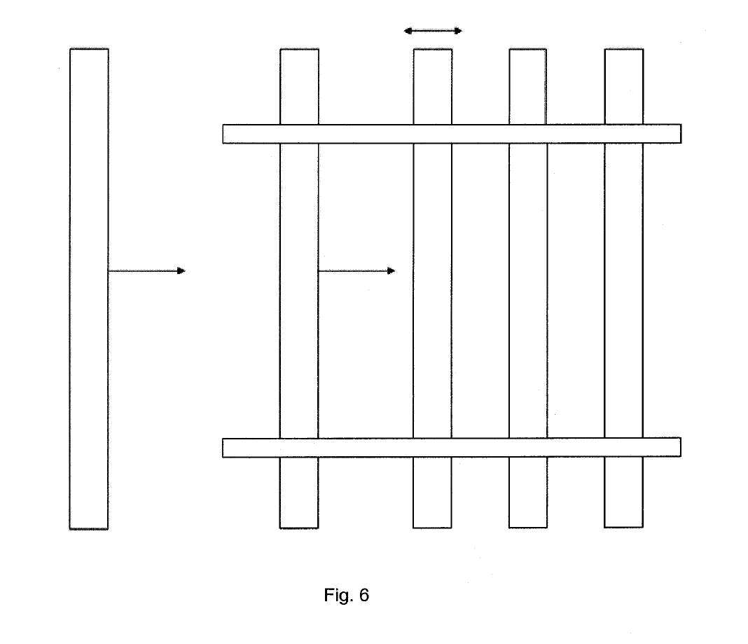

In alternate embodiments, a rackable blade may slide into place along a direction perpendicular to a longitudinal axis with respect to the blade, as shown in the top view provided by FIG. 6. For instance, a blade may be mounted by allowing it to slide along a rail, wheels, guides, configurations utilizing ball bearings, or any other mechanism known in the art. In some embodiments of the invention, when a blade is mounted, it may be locked into place. A rack may have pre-set locations where a blade may be mounted, such that a blade may slide into the proper place and then go no further.

In another embodiment of the invention, a blade may by placed to be supported by a rack using any other known method. For instance, a blade may be placed into the desired location and be clamped into place. Similarly, a blade can be placed into a desired location and be suspended by a hook, hanger, or other type of fixture.

In some embodiments, a blade may be fixed into a location once it is mounted. For example, once a blade slides into its spot, it may not move in other directions. In some cases a blade may be locked into place. In some cases, manual intervention, or some sort of automated process may be initiated in order to unlock a blade from its position. In some cases, automated ejection processes may partially or completely remove a blade from its mounted position.

In accordance with some embodiments, when a blade is mounted, its position along a rack may be adjustable. For example, as shown in FIGS. 5 and 6, a blade may move along a rack after it has been mounted from a direction parallel or perpendicular to a longitudinal axis with respect to the blade. In some embodiments, a blade may move along a rack through manual intervention, while in other embodiments, a blade may move along a rack through an automated process. The blades may interface with a backplane or other support structure in a manner that enables the blades to move in a predetermined manner, while maintaining the connection or communication with the backplane or other support structure.

A control system may control the placement of the blades with respect to one another for various applications. For instance, blade placement may be varied to adjust light exposure to the algae within the blades. Blade placement may also be adjusted to allow maintenance and inspection of the various blades. In some embodiments, a user may interact with a user interface that may instruct a control system to adjust the placement of the blades accordingly. In some embodiments, pre-programmed automated instructions may control the blade placement.

When a blade is mounted onto a rack, it may allow communications between the blade and a controls and monitoring system. In some embodiments sensors may be provided on the blade or on the rack that may provide feedback about the conditions of the blade. Such sensors may include sensors such as light sensors, temperature sensors, pH sensors, sensors to detect flow rate, pressure sensors, density sensors, sensors to determine O₂ level within the photobioreactor, sensors to monitor the CO₂ being provided to the photobioreactor, and sensors to track the growing process of the algae. Such sensors may communicate with a controls and monitoring system which may or may not adjust settings as desired.

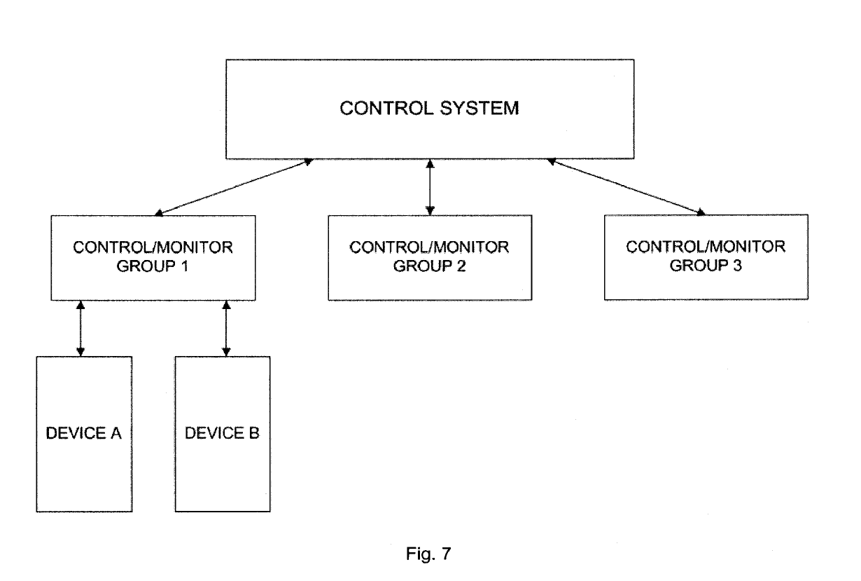

FIG. 7 shown an example of a control system in accordance with one aspect of the invention. In one embodiment, a central control system or scheme may be implemented. The control system may communicate with control/monitoring interfaces for one or more group. The control/monitoring interface for each group may communicate with one or more devices or groups of devices. A device or group of devices may be monitored through various sensors or transducers, such as those described.

For example, a photobioreactor site may include one or more groupings of photobioreactors with various hierarchies. In one example, one cluster may include three pods, each pod comprising six blades. Three clusters may make up one hive, and any number of hives may be provided at a site. Any number of photobioreactor devices or groupings or levels may be provided. Any of the devices or groupings may include a control/monitoring interface. For instance, each cluster may have a control-monitoring interface that may communicate with a central control system, and the cluster's control/monitoring interface may communicate with each of the pods, or with each of the blades within the cluster.

There may be multiple levels of control/monitoring interfaces. For instance, a pod may have a control/monitoring interface that may communicate with each of the blades of the pod. A control/monitoring interface of a cluster may communicate with the interface for the various pods. A control/monitoring interface for a hive may communicate with the interface for a cluster. The central control system may communicate with the interface for a hive.

A control/monitoring interface may monitor and/or control a device or a group of devices. For example, a control/monitoring interface for a pod may control or monitor the conditions for each of the blades of the pod, or may control or monitor them in groups, such as two groups of three pods.

In one embodiment, a central control system may monitor and control each device or group of devices without communicating with intermediate control/monitor groups. In an alternate embodiment, there may be no control system, and groups of devices may have their own control system without communication in between them. For instance, each hive may have its own control system.

The various control/monitoring interfaces or control systems may communicate with one another over a network. In some embodiments, the network may be a local area network, or a wide area network, or the Internet. The interfaces may be physically connected to one another or to a network through a wire, or may communicate wirelessly.

A user may be able to interact with a control system along any level of interaction. For example, a user may access a central control system and control or monitor the conditions relating to the photobioreactors at any level. In some embodiments, a user may access a central control system through a user interface, which may be provided by a computer, PDA, phone, laptop, or any other network device. In some embodiments, the user interface may be integrated with a structure that may be part of a photobioreactor apparatus.

A user may also be able to interact with a control/monitor group interface. For example, a user may be able to interact with a user interface that may be provided at a pod, which may or may not also communicate with a central control system.

A control system may monitor various conditions relating to photobioreaction. In some instances, when a sensor detects a condition that may be cause for concern, an alarm or alert may be provided to the control system, which may notify a user of a condition, or may adjust a setting to deal with the concern. For example, if a sensor detects that a blade has ceased to function, it may notify the central system, which may notify a user to repair or replace the blade. A system may also take independent action, such as utilizing another blade that may have been idle. In another example, an alarm may be provided if a sensor detects an alarming condition, such as a temperature that has risen extraordinarily high. The system may or may not take independent action to try to lower the temperature to an acceptable, or non-alarming level.

A control system may control the culture growth within a photobioreactor. In some embodiments, the control system may implement different control methods based on the type of algae grown. Different types of methods of culturing algae can be used. For example, in accordance with one aspect of the invention, the invention provides for a method of culturing algae or other growth based on a batch, continuous, fed-batch cultures, or any combination thereof.

In a growth method, algae may be injected into a photobioreactor. The algae may enter through one or more inlet. The algae may be injected with a growth medium. In some implementations, the algae and growth medium may be stored in a different container and may be provided from different sources, or may be injected from the same source. In some implementations, the algae may be injected at an early stage of growth. In other implementations, algae may be at a more advanced stage of growth when injected into a photobioreactor. In some situations, an algae may grow in a holding tank before being injected into a photobioreactor.

In a batch process, in accordance with one embodiment of the invention, an algae may be injected into a photobioreactor to execute growth. The algae may be injected at desired concentration levels. Then algae may be grown in exposed to desirable growth conditions (which may include, but are not limited to desirable levels of light, temperature, pH, CO₂ or O₂ levels) to allow for a desirable growth optimization. When growing, the algae may reach a stationary phase in which vegetative growth stops, but secondary metabolites may accumulate. In some cases, the desirable growth conditions may be adjusted in order to optimize the accumulation of desirable secondary metabolites. One example of a desirable secondary metabolite may be a lipid used to make a biofuel. In other cases, a photobioreactor unit, such a blade may be swapped from one location with desirable vegetative growth conditions to another location with desirable metabolite accumulation conditions. Such locations can include different racks with different control schemes or components. In some cases, a desirable by-product of growth, such as oxygen, may be released and/or collected during the growth process. The algae may then be harvested from the photobioreactor. In some cases, the algae may be harvested when a desirable metabolite has accumulated at a maximum level. In other cases, algae may be harvested when vegetative growth is maximized or when a system detects the algae is about to decline. The desirable metabolite may be isolated from the algae biomass, purified and concentrated.

In another example, in a continuous process, an algae may be injected into a photobioreactor more or less continuously and harvested more or less continuously. Different portions or regions of a photobioreactor may contain algae at different stages of growth. The growth conditions may be adjusted at the various portions or regions to optimize the desired growing conditions for the stage of growth. In some embodiments, an algae may be transported by a pump or fluid pressure. For example, near an inlet, algae may be at an earlier stage of growth, while near an outlet, an algae may have grown to accumulate a substantial amount of desirable metabolite.

In some implementations, such as fed batch cultures or other culture methods, nutrients to aid in desired growth may be provided to the photobioreactor. The nutrients may be added in desired amounts to support the desired amount or type of growth. In some instances, the same or different nutrients may be added at various portions or regions of a photobioreactor unit.

A control system may provide flow control for the various photobioreactor units. For example, the control system may monitor the inflow of CO₂ or various nutrients, and the release of O₂ from the unit. For instance, if the system detects that the level of CO₂ is too high or too low, it can adjust the inflow of CO₂ accordingly. The desired levels of CO₂, nutrients, and O₂ flow may be controlled to provide an optimum condition for algae growth. The control system may determine the optimum flow rates based on other conditions, information of which may be provided by other sensors, including, but not limited to, type of algae being grown, the current algae growth status, temperature, or light exposure.

In some embodiments, a CO₂ source or storage may be provided. The CO₂ source may be connected with a photobioreactor structure in any number of ways. CO₂ may be provided directly to each photobioreactor unit, and may be regulated by a central system. In another embodiment, CO₂ may be provided to any grouping of photobioreactor units, such as, for example, a pod. The grouping of units (i.e. the pod) may have a control interface that may receive the CO₂ from the source and control the CO₂ delivery to each unit of the grouping, without requiring communication with a central system.

Similarly, O₂ release may be monitored and controlled at a local level or at a central level. For instance, if the O₂ level is too high, O₂ release may be increased. Each photobioreactor unit may be individually controllable, or a group of photobioreactors may be controllable. O₂ that is released may be collected and stored, or sent to another location.

The various in or out-flows of a photobioreactor unit including, but not limited to, the flow of the algae, products, gases such as CO₂ or O₂, or nutrients may be caused my any mechanism known in the art. For instance, circulation may be induced by a positive pressure source, or a negative pressure source. Some examples may include a pump, such as a diaphragm pump, centrifugal pump, peristaltic pump, or any other pump known to those skilled in the art, or a vacuum source.

An alarm or alert system may also communicate with a user regarding the CO₂ or O₂ levels for a photobioreactor. For instance, if a level is too high or too low the system may try to adjust the level to fall within an acceptable range or tolerance. If the system detects that it may not be able to adjust a level at an acceptable rate, it may provide an alert to that effect. If the system is unable to adjust the level due to control failure or any other reason, it may provide an alert to that effect as well.

A control system may detect when a photobioreactor unit needs to be harvested. In some embodiments, a sensor may be provided to determine an optimal time for harvesting. In some cases, harvesting may occur when the sensor detects that it is a good time to harvest. Physical parameters indicating algae growth may be measured to determine a good harvest time. In alternate cases harvesting may be more or less continual. In other cases, harvesting may occur at set periods of time. The position of a photobioreactor may or may not be adjusted while harvesting occurs.

The control system may also detect when a photobioreactor unit needs to be cleaned. In some embodiments, when a control system detects that a unit needs to cleaned, it may use an automatic self-cleaning system that can take place without user intervention. In one embodiment, a cleaning surface may travel within a pipe of the photobioreactor unit to scrub the internal surface of the pipe. In another embodiment, a cleaning solution or gel may flow within a pipe to cleanse the interior. In some cases, bubbles may provide agitation to help clean the pipe. Additional examples of cleaning systems may be provided by PCT Publication No. WO 1994/009112, U.S. Pat. No. 5,242,827, and U.S. Pat. No. 6,370,815, which are hereby incorporated in their entirety. In some embodiments, when a control system detects a unit needs to be cleaned, it may automatically remove the unit to be cleaned and swap in a clean unit. The swapped out unit may undergo a cleaning process that may be external to the rack or support structure for the unit.

In an alternate embodiment, when a control system detects a photobioreactor unit needs to be cleaned, it may provide an alert. In such a situation, a user may clean the unit or remove the unit for cleaning.

In some situations a photobioreactor unit may be cleaned periodically. For instance a system may be configured such that every month, a unit is cleaned. A system may provide a staggered arrangement so that not all units are cleaned at once. For instance, a staggered arrangement may be arranged such that one unit within a grouping of units is cleaned every week, or that any number of units within a grouping of units is cleaned every week. A staggered arrangement may allow overall production to remain more or less constant.

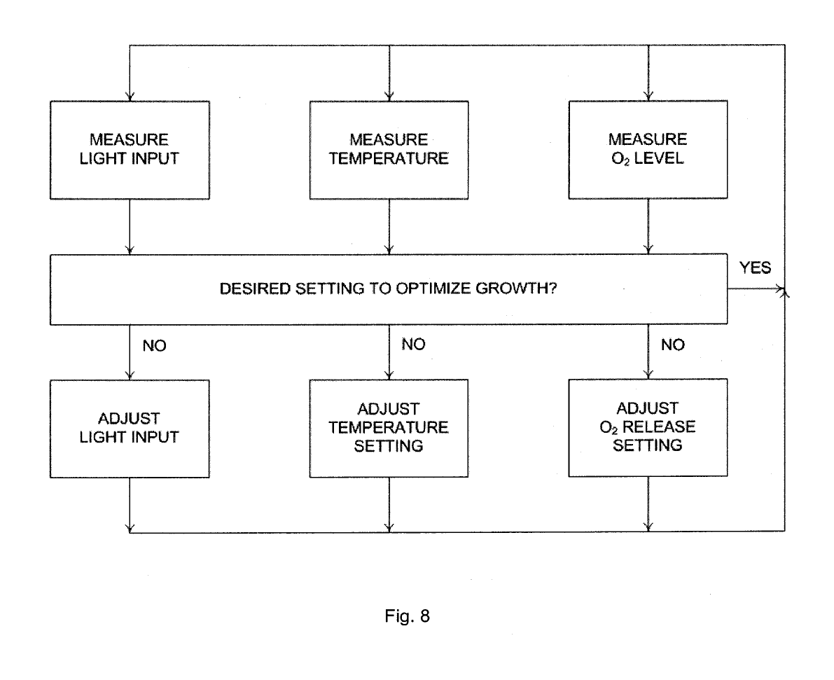

FIG. 8 shows an example of an optimization method in accordance with one aspect of the invention. As sensors may provide feedback to a control system, the control system may initiate adjustments as needed to optimize algae growing conditions. For example, sensors may measure light input to a photobioreactor unit, measure the temperature within a photobioreactor, or measure the O₂ level within the photobioreactor unit. In another example, a sensor may determine the concentration of algae, or a composition parameter of the culture. The control system may receive these measurements and determine whether they are at the desired setting to optimize algae growth. The desired settings may depend on a combination of factors, including the type of algae being grown, the current growth state of the algae, or the desired growth rate of the algae.

Based on the factors, a control scheme may be set, in which a desired light input, temperature, desired flow rate, or O₂ level may be set. If the measurements from the sensors match the desired settings, the system may do nothing other than maintain the settings and continue to monitor the settings. If the measurements from the sensors do not match the desired settings, then the settings may be adjusted accordingly. For instance, if the control system determines that light input is a little low, and that O₂ level is a little high, the control system may increase light input, and increase O₂ release. In some situations, a user may input the type of algae being grown, and a control scheme may be selected accordingly. Or a user may select a control scheme from one or more control schemes provided. In some situations, only one control scheme may be available for a system.

In some alternate embodiments, the system may operate as a closed-loop system without feedback. For instance, the desired settings may be specified, and the various equipment may be set to operate at the settings without receiving feedback on the effect of the settings. For instance, a light input and a heater setting may be specified and may operate on those conditions without determining what the actual light received or temperature at the photobioreactor unit are.

In some embodiments, there may be a control order preference, rather than a desired set point for each factor. For instance, the control system may determine that it is preferable to adjust light input, rather than adjusting the temperature. In the event that the factors are coupled such that adjusting one may affect the desired set point for another, the control system may adjust a preferred factor first. For instance, if it is preferable to adjust light, and a temperature is within an acceptable range, the control system may adjust the light input to optimize the system based on the conditions provided by the given temperature setting. If the system is coupled such that adjusting light will affect the temperature, the temperature may be adjusted as fine tuning after the light is adjusted to an optimal setting.

The various factors for growth may be regulated in accordance with the photobioreactor apparatus. For example, light input may be regulated by regulating a light source. For instance, the brightness, amount, or wavelength of the light source may be adjustable. In another example, the amount of time of exposure to a light source may be varied. The type of light source itself (i.e. using an LED vs. using an incandescent bulb) may be varied. Also, the positioning of the light source or photobioreactor unit may be varied. For example, light sources may be provided between vertically aligned blades. The position of the blade may be adjusted to increase or decrease light exposure. The photobioreactor may allow re-reflection of light that is emitted.

In some embodiments, a combination of natural light from the sun and artificial light sources may be utilized. The amount of light exposure may be decreased by providing shading or some sort of obstruction or layer between the light source and the photobioreactor unit. In some embodiments, one portion of a photobioreactor unit may be exposed to greater amounts of light for a given time period, and then another portion of the photobioreactor unit may be exposed to greater amount of lights for a time period. In some instances, light may irradiate through a portion of the photobioreactor. As increased amounts of growth occurs, the amount of light irradiating through may decrease and the light source may be adjusted accordingly.

In accordance with another embodiment of the invention, the temperature of a photobioreactor may be regulated. For example, a pipe, in which algae grows, may include a concentric pipe through which a fluid flows to regulate the temperature within the larger pipe. The temperature of the fluid flowing within the concentric pipe may be adjusted to regulate the temperature within the larger pipe of the photobioreactor. The temperature of the inner pipe may be regulated by heating or cooling the fluid within the inner pipe. The temperature of the inner pipe may also be regulated rapidly by switching the fluids within the inner pipe. For example, a first fluid source may have a hot fluid, and a second fluid source may have a cooler fluid, and to provide rapid cooling within the inner pipe, a fluid source may be switched from the first fluid source to the second fluid source. In some cases external heaters or cooling apparatus may also regulate the temperature within a photobioreactor. For instance, electric heaters, heat exchangers, or sprayers may be used.

In some embodiments, the temperature of a fluid within a concentric pipe may be a primary control and external heaters or cooling apparatus may provide a secondary control. External heaters or cooling apparatus may provide more localized temperature control—for instance, they may adjust the temperature of a portion of a photobioreactor unit. For example, if a photobioreactor unit is a blade consisting of a plurality of pipes, the heater/cooler may be directed to a portion of a subset of pipes.

In some cases, a light source may also provide heat, and adjusting a light source may or may not initiate a heat source adjustment.

The O₂ level may also be regulated in accordance with one embodiment of the invention. Means for O₂ release may be provided to a photobioreactor unit. In some situations, a photobioreactor unit may have one or more O₂ releasing mechanisms. In some embodiments, the control for O₂ release may be for a grouping of photobioreactor units. For example, O₂ may be collected from multiple-blades, but may be released on a pod basis.

For any factors or parameters associated with photobioreaction, an alarm or alert may be provided if a factor falls outside an acceptable tolerance. Similarly, an alarm or alert may be provided if a sensor malfunction is registered. Additionally, if a factor can not be brought into an acceptable tolerance within an acceptable amount of time or rate, an alarm or alert may be provided while the system regulates the factor towards the tolerance. If the system detects that a seriously alarming condition exists (e.g., a rapid drop in pressure indicating a pipe has ruptured), the system may automatically shut down one or more unit. If one unit is shut down, other units may continue to operate.

Any of the methods and apparatus described herein may be used with other photobioreactor systems such, as those described in U.S. Provisional Patent Application No. 61/106,962, filed Oct. 20, 2008, which is herein incorporated by reference in its entirety.

It should be understood from the foregoing that, while particular implementations have been illustrated and described, various modifications can be made thereto and are contemplated herein. It is also not intended that the invention be limited by the specific examples provided within the specification. While the invention has been described with reference to the aforementioned specification, the descriptions and illustrations of the preferable embodiments herein are not meant to be construed in a limiting sense. Furthermore, it shall be understood that all aspects of the invention are not limited to the specific depictions, configurations or relative proportions set forth herein which depend upon a variety of conditions and variables. Various modifications in form and detail of the embodiments of the invention will be apparent to a person skilled in the art. It is therefore contemplated that the invention shall also cover any such modifications, variations and equivalents.

What is claimed is:

1. A method for operating a photobioreactor system comprising:
selecting a plurality of photobioreactor units; and
sliding the plurality of photobioreactor units into a rack support structure for growing microorganisms.

2. A method comprising:
monitoring operational conditions for a plurality of rack-mounted photobioreactor units;
providing an interface configured to communicate information regarding the operational conditions to a user,
wherein the interface is configured to communicate an alarm or alert if one is provided.

3. A method of optimizing growth conditions comprising:
determining a plurality of desired operating setting to optimize a growth condition using a photobioreactor backplane interface;
measuring operating conditions of a plurality of photobioreactors with the photobioreactor backplane interface;
determining whether the operating conditions are within a specified tolerance of the desired operating settings; and
adjusting an operating condition to regulate towards or fall within the specified tolerance of the desired operating setting if the operating condition does not initially fall within the specified tolerance.

4. The method of claim 3 wherein the operating conditions includes at least one of the following: light input to the photobioreactor, temperature of the photobioreactor, O₂ levels within the photobioreactor, or CO₂ inflow rate.
